(19) United States
(12) Patent Application Publication
(10) Pub. No.: US 2015/0133343 A1
(43) Pub. Date: May 14, 2015

(54) POLYPEPTIDE MARKERS FOR THE DIAGNOSIS OF PROSTATE CANCER

(71) Applicant: Mosaiques Diagnostics and Therapeutics AG, Hannover (DE)

(72) Inventor: Harald Mischak, Sehnde (DE)

(21) Appl. No.: 14/584,146

(22) Filed: Dec. 29, 2014

Related U.S. Application Data
(63) Continuation of application No. 11/887,562, filed on Oct. 1, 2007, now abandoned.

(30) Foreign Application Priority Data
Apr. 7, 2005 (EP) 05102741.5
Jun. 21, 2005 (EP) 05105434.4

(51) Int. Cl. G01N 33/68; G01N 33/574

(52) U.S. Cl. CPC G01N 33/6848; G01N 33/57434
USPC 506/12

(57) ABSTRACT

A method for the diagnosis of prostate cancer, comprising the step of determining the presence or absence of at least three polypeptide markers in a sample, wherein the polypeptide marker is selected from markers 1 to 44 and 52 to 78 (frequency markers), or determining the amplitude of at least one polypeptide marker selected from markers 45 to 51 and 79 to 115 (amplitude markers), wherein said sample is a urine sample or seminal fluid sample.

### CROSS-REFERENCED APPLICATION

This application is a continuation of, and claims priority to and the benefit of U.S. patent application Ser. No. 11/887,562, filed Oct. 1, 2007, and incorporates the disclosure of that application as if fully set forth herein in its entirety. Serial No.:

[symbol]

### BACKGROUND 1. Field of the Disclosure

The present disclosure relates to the use of the presence or absence of one or more peptide markers in a sample from a subject for the diagnosis of prostate cancer (PCA) and to a method for the diagnosis of prostate cancer, wherein the presence or absence of the peptide marker or markers is indicative of the existence of prostate cancer.

2. Discussion of the Background Art

The carcinoma of the prostate gland is one of the most common cancers in males. Since complaints occur only in a stage of advanced disease, the cancer can be diagnosed in an early stage only by regular screening tests for early detection (digital rectum exam and PSA (prostate specific antigen) value in the blood). To confirm the suspicion diagnosis, a tissue specimen is withdrawn by means of fine-needle biopsy.

For the therapy, there are several possibilities that depend on the kind and stage of the tumor and on the individual needs of the patient: In an early stage, seed implantation (minimal-invasive introduction of iodine-125 radioactive emitters into the prostate) or surgical removal of the tumor and irradiation from outside are available.

At the time of diagnosis, metastasizing into other organs has already occurred in one third of the patients; at this time, the disease can hardly be healed any more. However, radiotherapy, chemotherapy or hormone therapy (combinations are possible) can delay the further spreading of the cancer. When the prostate gland is affected in isolation, i.e., metastasizing has not occurred yet, the prognosis is favorable.

A benign tumor (adenoma) of the prostate gland, benign prostate hyperplasia (BPH), occurs much more frequently than the carcinoma. According to the prior art, it can be distinguished from a malignant carcinoma only very unreliably by means of the PSA value. In this case too, a biopsy must be performed to be able to make a clear diagnosis.

As described above, there is no non-invasive and reliable early detection of prostate cancer. To date, a clear diagnosis has been associated with invasive operations, such as biopsy. Thus, there has been a need to find a process and method for a diagnosis of prostate cancer that is as little as possible invasive, quick and inexpensive.

WO 03/072710 describes protein biomarkers for distinguishing between prostate carcinoma cells and BPH. The application describes markers that have high uncertainties (in a range of more than ±0.5%). Due to the imprecise information, an assignment to individual markers is virtually impossible from the large number of molecules in this range. A cell lysate of prostate epithelial cells is preferably employed as a sample material, which requires a complicated sampling procedure. Due to the large number of proteins included in the definition and the difficulty of sampling, the method is little suitable.

WO 01/25791 describes methods for the diagnosis of prostate carcinomas using markers. The mentioned markers are derived from the protein semenogelin I. The stated masses are defined to a certainty of only ±0.5%. Studies show that the mentioned markers are unsuitable.

Surprisingly, it has now been found that particular peptide markers in a sample from a subject can be used for the diagnosis of prostate cancer and for the differential diagnosis to distinguish between prostate cancer and benign prostate hyperplasia (BPH). In particular, the samples may be urine or seminal fluid samples, which are withdrawn non-invasively.

### SUMMARY OF THE DISCLOSURE

Consequently, the present disclosure relates to the use of the presence or absence of at least three polypeptide markers in a sample from a subject for the diagnosis of prostate cancer, wherein said polypeptide marker is selected from polypeptide marker Nos. 1 to 115 as characterized by the molecular masses and migration times as stated in Table 1.

TABLE 1
Polypeptide markers for the diagnosis of prostate cancer (PCA) and their
molecular masses and migration times (CE time in minutes):

| | | CE time |
|---|---|---|
| Number | | [min] |
| | Mass (Da) | |
| 1 | 1579.7 | 26.4 |
| 2 | 1991.9 | 17.1 |
| 3 | 1955.9 | 24.8 |
| 4 | 1140.5 | 21.4 |
| 5 | 1677.3 | 7.4 |
| 6 | 10753.1 | 13.6 |
| 7 | 1412.6 | 22.1 |
| 8 | 1636.8 | 27.2 |
| 9 | 1946.9 | 28.4 |
| 10 | 5887.6 | 25.1 |
| 11 | 12407.9 | 22.2 |
| 12 | 1186.6 | 17.4 |
| 13 | 1240.6 | 23.3 |
| 14 | 1326.6 | 24.8 |
| 15 | 3802.1 | 30.1 |
| 16 | 1749.9 | 19.4 |
| 17 | 2216.1 | 31.0 |
| 18 | 1069.5 | 22.7 |
| 19 | 1341.6 | 26.6 |
| 20 | 1353.7 | 21.8 |
| 21 | 1716.8 | 24.6 |
| 22 | 2939.1 | 31.1 |
| 23 | 3002.0 | 19.2 |
| 24 | 1110.4 | 31.5 |
| 25 | 1496.7 | 26.5 |
| 26 | 1825.8 | 28.4 |
| 27 | 1180.5 | 33.9 |
| 28 | 2421.1 | 32.5 |
| 29 | 4345.9 | 30.6 |
| 30 | 2077.1 | 16.9 |
| 31 | 1134.6 | 19.4 |
| 32 | 1444.8 | 13.5 |
| 33 | 1046.5 | 21.9 |
| 34 | 1992.2 | 16.8 |
| 35 | 4069.6 | 21.3 |
| 36 | 1191.5 | 34.5 |
| 37 | 1239.5 | 32.5 |
| 38 | 2191.9 | 17.2 |
| 39 | 1865.8 | 30.0 |
| 40 | 1265.6 | 23.4 |
| 41 | 1936.1 | 10.5 |
| 42 | 911.3 | 31.9 |
| 43 | 1494.7 | 26.4 |
| 44 | 1209.6 | 22.5 |
| 45 | 1193.3 | 34.2 |
| 46 | 1235.4 | 34.3 |
| 47 | 1390.5 | 35.0 |
| 48 | 2292.1 | 23.7 |
| 49 | 2736.3 | 17.1 |
| 50 | 3385.5 | 21.0 |
| 51 | 3945.2 | 21.4 |
| | Masses (Da) | |
| 52 | 1636.8 | 27.2 |
| 53 | 1412.6 | 22.1 |
| 54 | 1579.7 | 26.4 |
| 55 | 1390.5 | 35.0 |
| 56 | 1196.6 | 15.8 |
| 57 | 11041.4 | 16.6 |
| 58 | 2971.4 | 17.9 |
| 59 | 3136.6 | 20.0 |
| 60 | 4409.9 | 14.2 |
| 61 | 1494.7 | 26.4 |
| 62 | 1833.9 | 24.2 |
| 63 | 2752.4 | 14.0 |
| 64 | 3515.8 | 15.3 |
| 65 | 1082.5 | 17.7 |
| 66 | 1878.9 | 15.3 |
| 67 | 3593.4 | 14.5 |
| 68 | 1236.7 | 13.3 |
| 69 | 2736.3 | 17.1 |
| 70 | 2973.4 | 20.0 |
| 71 | 1134.6 | 19.4 |
| 72 | 2191.9 | 17.2 |
| 73 | 2205.1 | 25.1 |
| 74 | 3959.7 | 14.0 |
| 75 | 1749.9 | 19.4 |
| 76 | 4069.6 | 21.3 |
| 77 | 2816.3 | 24.7 |
| 78 | 3435.8 | 15.0 |
| | Mass (Da) | |
| 79 | 973.26 | 35.44 |
| 80 | 1128.54 | 25.66 |
| 81 | 1173.58 | 37.47 |
| 82 | 1184.6 | 26.43 |
| 83 | 1290.39 | 30.85 |
| 84 | 1338.66 | 23.96 |
| 85 | 1428.45 | 36.73 |
| 86 | 1450.6 | 37.47 |
| 87 | 1460.71 | 19.83 |
| 88 | 1498.46 | 35.31 |
| 89 | 1526.76 | 23.54 |
| 90 | 1588.77 | 30.24 |
| 91 | 1675.77 | 29.23 |
| 92 | 1703.91 | 33.67 |
| 93 | 1808.87 | 23.72 |
| 94 | 1863.96 | 44.01 |
| 95 | 1867.79 | 33.29 |
| 96 | 1925.9 | 23.2 |
| 97 | 2036.97 | 31.53 |
| 98 | 2114.05 | 31.59 |
| 99 | 2196.11 | 33.16 |
| 100 | 2212.06 | 33.36 |
| 101 | 2257.95 | 36.02 |
| 102 | 2544.06 | 26.14 |
| 103 | 2599.21 | 28.05 |
| 104 | 2761.38 | 21.47 |
| 105 | 3334.17 | 31.01 |
| 106 | 3361.41 | 24.32 |
| 107 | 3426.43 | 27.73 |
| 108 | 3765.51 | 20.18 |
| 109 | 3864.56 | 33.82 |
| 110 | 3870.8 | 33.47 |
| 111 | 4252.03 | 28.77 |
| 112 | 6211.93 | 20.27 |
| 113 | 6650.86 | 25.55 |
| 114 | 6820.37 | 21.03 |
| 115 | 16918.24 | 19.8 |

With the present disclosure, it is possible to diagnose prostate cancer at a very early stage. Thus, the disease can be cured by known methods at an early stage. The disclosure further enables an inexpensive, quick and reliable diagnosis with in part non-invasive or only minimal-invasive operations.

The disclosure further relates to differential diagnosis for distinguishing between prostate carcinoma and BPH. The differential diagnosis can be attained by using the presence or absence of at least three polypeptide markers in a sample from a subject, wherein said polypeptide marker is selected from polypeptide markers 52 to 78, which are characterized by the molecular masses and migration times as stated in Table 2. Preferably, more markers are employed.

TABLE 2
Polypeptide markers for the differential diagnosis of prostate cancer or
BPH, their molecular masses and migration times.

| Number | Masses (Da) | CE time [min] |
|---|---|---|
| 52 | 1636.8 | 27.2 |
| 53 | 1412.6 | 22.1 |
| 54 | 1579.7 | 26.4 |
| 55 | 1390.5 | 35.0 |
| 56 | 1196.6 | 15.8 |
| 57 | 11041.4 | 16.6 |
| 58 | 2971.4 | 17.9 |
| 59 | 3136.6 | 20.0 |
| 60 | 4409.9 | 14.2 |
| 61 | 1494.7 | 26.4 |
| 62 | 1833.9 | 24.2 |
| 63 | 2752.4 | 14.0 |
| 64 | 3515.8 | 15.3 |
| 65 | 1082.5 | 17.7 |
| 66 | 1878.9 | 15.3 |
| 67 | 3593.4 | 14.5 |
| 68 | 1236.7 | 13.3 |
| 69 | 2736.3 | 17.1 |
| 70 | 2973.4 | 20.0 |
| 71 | 1134.6 | 19.4 |
| 72 | 2191.9 | 17.2 |
| 73 | 2205.1 | 25.1 |
| 74 | 3959.7 | 14.0 |
| 75 | 1749.9 | 19.4 |
| 76 | 4069.6 | 21.3 |
| 77 | 2816.3 | 24.7 |
| 78 | 3435.8 | 15.0 |

The migration time is determined by capillary electrophoresis (CE), for example, as set forth in the Example under item 2. In this Example, a glass capillary of 90 cm in length and with an inner diameter (ID) of 50 μm and an outer diameter (OD) of 360 μm is operated at an applied voltage of 30 kV. As the mobile solvent, 30% methanol, 0.5% formic acid in water is used.

It is known that the CE migration times may vary. Nevertheless, the order in which the polypeptide markers are eluted is typically the same under the stated conditions for any CE system employed. In order to balance any differences in the migration time that may nevertheless occur, the system can be normalized using standards for which the migration times are exactly known. These standards may be, for example, the polypeptides stated in the Examples (see the Example, item 3).

The characterization of the polypeptides shown in Tables 1 to 2 was determined by means of capillary electrophoresis-mass spectrometry (CE-MS), a method which has been described in detail, for example, by Neuhoff et al. (Rapid communications in mass spectrometry, 2004, Vol. 20, pages 149-156). The variation of the molecular masses between individual measurements or between different mass spectrometers is relatively small when the calibration is exact, typically within a range of ±0.03%, preferably within a range of ±0.01%.

### BRIEF DESCRIPTION OF THE DRAWINGS

FIG. 1 is a table which lists the large number of sequences in accordance with the present disclosure;

FIG. 2 is a graph showing the amplitudes of six markers;

FIG. 3a is a graph plotting sensitivity vs. 100-specificity for six markers that are described in WO 01/25791 and could be found in urine samples;

FIG. 3b is a graph plotting sensitivity vs. 100-specificity for the biomarkers according to the disclosure with Nos. 1 to 78 of the present patent application;

FIG. 3c is a graph plotting sensitivity vs. 100-specificity the markers according to the disclosure (16 markers); and

FIG. 3d is a graph plotting sensitivity vs. 100-specificity for markers 24, 41 and 46.

### DETAILED DESCRIPTION OF THE PREFERRED EMBODIMENT

The polypeptide markers according to the disclosure are proteins or peptides or degradation products of proteins or peptides. They may be chemically modified, for example, by posttranslational modifications, such as glycosylation, phosphorylation, alkylation or disulfide bridges, or by other reactions, for example, within the scope of degradation. In addition, the polypeptide markers may also be chemically altered, for example, oxidized, during the purification of the samples.

Proceeding from the parameters that determine the polypeptide markers (molecular weight and migration time), it is possible to identify the sequence of the corresponding polypeptides by methods known in the prior art.

The polypeptides according to the disclosure (see Tables 1 to 5) are used to diagnose prostate cancer. “Diagnosis” means the process of knowledge gaining by assigning symptoms or phenomena to a disease or injury. In the present case, the existence of prostate cancer is concluded from the presence or absence of particular polypeptide markers. Thus, the polypeptide markers according to the disclosure are determined in a sample from a subject, wherein its presence or absence allows the conclusion of the existence of prostate cancer in the case of frequency markers. The presence or absence of a polypeptide marker can be measured by any method known in the prior art. Methods which may be used are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or defined from experience.

In the context of the present disclosure, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of prostate cancer (frequency markers). Thus, there are polypeptide markers which are typically present in patients with prostate cancer (ill), such as polypeptide markers No. 1 to 11, but absent in subjects with no prostate cancer (control). In addition, there are polypeptide markers which are present in subjects with no prostate cancer, but are less frequently or not at all present in subjects with prostate cancer, for example, Nos. 12 to 44 (Table 3).

TABLE 3
Polypeptide markers (frequency markers) for the diagnosis of prostate
cancer (PCA), their molecular masses and migration times as well as their
presence and absence in groups of patients suffering from prostate cancer
and control groups as a factor (1 = 100%, 0 = 0%; sample processing
and measurement as described in the Example).

| | | CE time | | Factor | Factor control |
|---|---|---|---|---|---|
| Number | Mass [Da] | [min] | Difference | PCA group | group |
| 1 | 1579.7 | 26.4 | D:0.75 | 1 | 0.25 |
| 2 | 1991.9 | 17.1 | D: 0.66 | 0.88 | 0.22 |
| 3 | 1955.9 | 24.8 | D: 0.65 | 1 | 0.35 |
| 4 | 1140.5 | 21.4 | D: 0.57 | 1 | 0.43 |
| 5 | 1677.3 | 7.4 | D: 0.57 | 0.63 | 0.06 |
| 6 | 10753.1 | 13.6 | D: 0.54 | 0.88 | 0.33 |
| 7 | 1412.6 | 22.1 | D: 0.53 | 0.75 | 0.22 |
| 8 | 1636.8 | 27.2 | D: 0.53 | 0.63 | 0.1 |
| 9 | 1946.9 | 28.4 | D: 0.53 | 0.75 | 0.22 |
| 10 | 5887.6 | 25.1 | D: 0.51 | 1 | 0.49 |
| 11 | 12407.9 | 22.2 | D: 0.51 | 0.75 | 0.24 |
| 12 | 1186.6 | 17.4 | D: −0.51 | 0.38 | 0.88 |
| 13 | 1240.6 | 23.3 | D: −0.51 | 0.25 | 0.76 |
| 14 | 1326.6 | 24.8 | D: −0.51 | 0.38 | 0.88 |
| 15 | 3802.1 | 30.1 | D: −0.51 | 0.38 | 0.88 |
| 16 | 1749.9 | 19.4 | D: −0.52 | 0.13 | 0.65 |
| 17 | 2216.1 | 31.0 | D: −0.52 | 0.13 | 0.65 |
| 18 | 1069.5 | 22.7 | D: −0.53 | 0 | 0.53 |
| 19 | 1341.6 | 26.6 | D: −0.53 | 0 | 0.53 |
| 20 | 1353.7 | 21.8 | D: −0.53 | 0.25 | 0.78 |
| 21 | 1716.8 | 24.6 | D: −0.53 | 0.25 | 0.78 |
| 22 | 2939.1 | 31.1 | D: −0.53 | 0.38 | 0.9 |
| 23 | 3002.0 | 19.2 | D: −0.53 | 0.25 | 0.78 |
| 24 | 1110.4 | 31.5 | D: −0.54 | 0.13 | 0.67 |
| 25 | 1496.7 | 26.5 | D: −0.54 | 0.13 | 0.67 |
| 26 | 1825.8 | 28.4 | D: −0.54 | 0.13 | 0.67 |
| 27 | 1180.5 | 33.9 | D: −0.55 | 0 | 0.55 |
| 28 | 2421.1 | 32.5 | D: −0.55 | 0 | 0.55 |
| 29 | 4345.9 | 30.6 | D: −0.55 | 0 | 0.55 |
| 30 | 2077.1 | 16.9 | D: −0.56 | 0.13 | 0.69 |
| 31 | 1134.6 | 19.4 | D: −0.57 | 0.25 | 0.82 |
| 32 | 1444.8 | 13.5 | D: −0.58 | 0.13 | 0.71 |
| 33 | 1046.5 | 21.9 | D: −0.59 | 0 | 0.59 |
| 34 | 1992.2 | 16.8 | D: −0.59 | 0 | 0.59 |
| 35 | 4069.6 | 21.3 | D: −0.59 | 0 | 0.59 |
| 36 | 1191.5 | 34.5 | D: −0.61 | 0 | 0.61 |
| 37 | 1239.5 | 32.5 | D: −0.61 | 0 | 0.61 |
| 38 | 2191.9 | 17.2 | D: −0.61 | 0.25 | 0.86 |
| 39 | 1865.8 | 30.0 | D: −0.62 | 0.13 | 0.75 |
| 40 | 1265.6 | 23.4 | D: −0.65 | 0.25 | 0.9 |
| 41 | 1936.1 | 10.5 | D: −0.65 | 0 | 0.65 |
| 42 | 911.3 | 31.9 | D: −0.69 | 0 | 0.69 |
| 43 | 1494.7 | 26.4 | D: −0.70 | 0.13 | 0.82 |
| 44 | 1209.6 | 22.5 | D: −0.71 | 0 | 0.71 |

In addition or also alternatively to the frequency markers (determination of presence or absence), the amplitude markers as stated in Table 4 may also be used for the diagnosis of prostate cancer (Nos. 45-51, 79-115). Amplitude markers are used in such a way that the presence or absence is not critical, but the height of the signal (the amplitude) decides if the signal is present in both groups. In Table 5, the mean amplitudes of the corresponding signals (characterized by mass and migration time) averaged over all samples measured are stated. To achieve comparability between differently concentrated samples or different measuring methods, all peptide signals of a sample are normalized to a total amplitude of 1 million counts. Therefore, the respective mean amplitudes of the individual markers are stated as parts per million (ppm). All groups employed consist of at least 20 individual patient or control samples in order to obtain a reliable mean amplitude. The decision for a diagnosis (PCA or not) is made as a function of how high the amplitude of the respective polypeptide markers in the patient sample is in comparison with the mean amplitudes in the control groups of the PCA group. If the amplitude rather corresponds to the mean amplitudes of the PCA group, the existence of PCA is to be considered, and if it rather corresponds to the mean amplitudes of the control group, the non-existence of PCA is to be considered. A more exact definition shall be given by means of marker No. 48 (Table 4). The mean amplitude of the marker is significantly increased in PCA (2600 ppm vs. 1600 ppm in the control group). Now, if the value for this marker in a patient sample is from 0 to 1600 ppm or exceeds this range by a maximum of 20%, i.e., from 0 to 1920 ppm, then this sample belongs to the control group. If the value is 2600 ppm or up to 20% below, or higher, i.e., between 2080 and very high values, the existence of PCA is to be considered.

The smaller the distance between the amplitudes of the control group and the PCA group, the closer a value that lies between the two reference values has to be to one of the reference values.

One possibility is to subdivide the range between the mean amplitudes into three portions. If the value is in the lower third, this is indicative of the lower value; if the value is in the upper third, this is indicative of the upper value. If it is in the middle third, a definite statement about this marker is not possible.

TABLE 4
Amplitude markers

| | | | | Mean amplitude in |
|---|---|---|---|---|
| | | CE time | Mean amplitude in | control samples |
| Number | | [min] | PCA samples [ppm] | [ppm] |
| | Mass (Da) | | | |
| 45 | 1193.3 | 34.2 | 24000 | 11000 |
| 46 | 1235.4 | 34.3 | 160 | 370 |
| 47 | 1390.5 | 35.0 | 22000 | 12000 |
| 48 | 2292.1 | 23.7 | 2600 | 1600 |
| 49 | 2736.3 | 17.1 | 120 | 350 |
| 50 | 3385.5 | 21.0 | 2600 | 1700 |
| 51 | 3945.2 | 21.4 | 260 | 120 |
| | Masses | | | |
| | (Da) | | | |
| 79 | 973.26 | 35.44 | 112.27 | 94.56 |
| 80 | 1128.54 | 25.66 | 104.1 | 135.92 |
| 81 | 1173.58 | 37.47 | 163.08 | 174.37 |
| 82 | 1184.6 | 26.43 | 61.33 | 53.82 |
| 83 | 1290.39 | 30.85 | 425.14 | 360.22 |
| 84 | 1338.66 | 23.96 | 116.85 | 223.77 |
| 85 | 1428.45 | 36.73 | 2325.09 | 1972.91 |
| 86 | 1450.6 | 37.47 | 348.86 | 350.46 |
| 87 | 1460.71 | 19.83 | 204.2 | 216.45 |
| 88 | 1498.46 | 35.31 | 115.26 | 94.66 |
| 89 | 1526.76 | 23.54 | 94.2 | 155.52 |
| 90 | 1588.77 | 30.24 | 504.83 | 565.2 |
| 91 | 1675.77 | 29.23 | 104.37 | 83.95 |
| 92 | 1703.91 | 33.67 | 326.26 | 495.27 |
| 93 | 1808.87 | 23.72 | 189.86 | 198.11 |
| 94 | 1863.96 | 44.01 | 989.35 | 1411.72 |
| 95 | 1867.79 | 33.29 | 117.97 | 108.75 |
| 96 | 1925.9 | 23.2 | 148.34 | 145.13 |
| 97 | 2036.97 | 31.53 | 74.45 | 77.29 |
| 98 | 2114.05 | 31.59 | 135.48 | 121.91 |
| 99 | 2196.11 | 33.16 | 479.36 | 390.89 |
| 100 | 2212.06 | 33.36 | 336.96 | 397.62 |
| 101 | 2257.95 | 36.02 | 365.38 | 567.62 |
| 102 | 2544.06 | 26.14 | 77.74 | 121.23 |
| 103 | 2599.21 | 28.05 | 797.34 | 915.08 |
| 104 | 2761.38 | 21.47 | 744.83 | 616.6 |
| 105 | 3334.17 | 31.01 | 105.87 | 127.64 |
| 106 | 3361.41 | 24.32 | 143.28 | 108.31 |
| 107 | 3426.43 | 27.73 | 149.46 | 116.27 |
| 108 | 3765.51 | 20.18 | 375.75 | 309.53 |
| 109 | 3864.56 | 33.82 | 271.44 | 162.02 |
| 110 | 3870.8 | 33.47 | 71.63 | 78.31 |
| 111 | 4252.03 | 28.77 | 388.07 | 447.93 |
| 112 | 6211.93 | 20.27 | 492.21 | 400.75 |
| 113 | 6650.86 | 25.55 | 233.38 | 232.51 |
| 114 | 6820.37 | 21.03 | 258.88 | 227.62 |
| 115 | 16918.24 | 19.8 | 605.64 | 324.52 |

For the differential diagnosis between PCA and BPH, Table 5 shows polypeptide markers that are typically present in patients with prostate carcinoma, such as the markers No. 52 to 58, but are not or rarely present in subjects with BPH. Further, there are polypeptide markers that are present in subjects with BPH, but occur less frequently or not at all in subjects with PCA, for example, the polypeptide markers No. 59 to 78.

TABLE 5
Polypeptide markers (frequency markers) for the differential diagnosis
between prostate cancer (PCA) and BPH, their molecular masses and
migration times as well as their presence and absence in groups of
patients suffering from prostate cancer as well as the BPH group as a
factor (1 = 100%, 0 = 0%; sample processing and
measurement as described in the Example).

| | Masses | CE time | | Factor of PCA | Factor of BP |
|---|---|---|---|---|---|
| Number | (Da) | [min] | Difference | group | group |
| 52 | 1636.8 | 27.2 | D: 0.63 | 0.63 | 0 |
| 53 | 1412.6 | 22.1 | D: 0.57 | 0.75 | 0.18 |
| 54 | 1579.7 | 26.4 | D: 0.55 | 1 | 0.45 |
| 55 | 1390.5 | 35.0 | D: 0.53 | 0.63 | 0.09 |
| 56 | 1196.6 | 15.8 | D: 0.51 | 0.88 | 0.36 |
| 57 | 11041.4 | 16.6 | D: 0.51 | 0.88 | 0.36 |
| 58 | 2971.4 | 17.9 | D: 0.50 | 0.5 | 0 |
| 59 | 3136.6 | 20.0 | D: −0.50 | 0.5 | 1 |
| 60 | 4409.9 | 14.2 | D: −0.50 | 0.5 | 1 |
| 61 | 1494.7 | 26.4 | D: −0.51 | 0.13 | 0.64 |
| 62 | 1833.9 | 24.2 | D: −0.51 | 0.13 | 0.64 |
| 63 | 2752.4 | 14.0 | D: −0.51 | 0.13 | 0.64 |
| 64 | 3515.8 | 15.3 | D: −0.51 | 0.13 | 0.64 |
| 65 | 1082.5 | 17.7 | D: −0.53 | 0.38 | 0.91 |
| 66 | 1878.9 | 15.3 | D: −0.53 | 0.38 | 0.91 |
| 67 | 3593.4 | 14.5 | D: −0.53 | 0.38 | 0.91 |
| 68 | 1236.7 | 13.3 | D: −0.55 | 0 | 0.55 |
| 69 | 2736.3 | 17.1 | D: −0.55 | 0 | 0.55 |
| 70 | 2973.4 | 20.0 | D: −0.55 | 0 | 0.55 |
| 71 | 1134.6 | 19.4 | D: −0.57 | 0.25 | 0.82 |
| 72 | 2191.9 | 17.2 | D: −0.57 | 0.25 | 0.82 |
| 73 | 2205.1 | 25.1 | D: −0.57 | 0.25 | 0.82 |
| 74 | 3959.7 | 14.0 | D: −0.57 | 0.25 | 0.82 |
| 75 | 1749.9 | 19.4 | D: −0.60 | 0.13 | 0.73 |
| 76 | 4069.6 | 21.3 | D: −0.64 | 0 | 0.64 |
| 77 | 2816.3 | 24.7 | D: −0.73 | 0 | 0.73 |
| 78 | 3435.8 | 15.0 | D: −0.82 | 0 | 0.82 |

The subject from whom the sample in which the presence or absence or the amplitude of one or more polypeptide markers is determined is derived may be any subject who is capable of suffering from prostate cancer, for example, an animal or human. Preferably, the subject is a mammal, such as a dog or a horse, and most preferably, it is a human.

For the application of the disclosure, not just one polypeptide marker, but a combination of markers is used to diagnose prostate cancer, wherein the existence of prostate cancer is concluded from their presence or absence and/or the height of the amplitude. By comparing a plurality of polypeptide markers, a bias in the overall result from a few individual deviations from the typical presence probability in the sick or control individual can be reduced or avoided.

The sample in which the presence or absence or amplitude of the polypeptide marker or markers according to the disclosure is measured may be any sample which is obtained from the body of the subject. The sample is a sample which has a polypeptide composition suitable for providing information about the state of the subject (prostate cancer or not). For example, it may be urine, sperm, seminal fluid (sperm without spermatozoa). Preferably, it is a liquid sample.

In a preferred embodiment, the sample is a urine sample.

Urine samples can be taken as known in the prior art. Preferably, a midstream urine sample is used in the context of the present disclosure. For example, the urine sample may be taken by means of a catheter or also by means of an urination apparatus as described in WO 01/74275.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands.

If required or desirable, the sample from the subject, for example, the urine sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, or electrophoretic separation. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the disclosure, the sample, before being measured, is separated by capillary electrophoresis, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kDa protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas the matrix-assisted laser desorption/ionization (MALDI) technique is used for measuring ions from a sample crystallized with a matrix. For analyzing the ions formed, quadrupoles, ion traps or time-of-flight (TOF) analyzers may be used.

In electrospray ionization (ESI), the molecules present in solution are atomized, inter alia, under the influence of high voltage (e.g., 1-8 kV), which forms charged droplets that become smaller from the evaporation of the solvent. Finally, so-called Coulomb explosions cause the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a very high resolution.

Preferred methods for the determination of the presence and absence of polypeptide markers include gas-phase ion spectrometry, such as laser desorption/ionization mass spectrometry, MALDI-TOF MS, SELDI-TOF MS (surface-enhanced laser desorption/ionization), LC-MS (liquid chromatography/mass spectrometry), 2D-PAGE/MS and capillary electrophoresis-mass spectrometry (CE-MS). All methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al., Journal of Chromatography A, 2003, Vol. 1013: 157-171, and Electrophoresis, 2004, 25: 2044-2055, and in Wittke et al., Journal of Chromatography A, 2003, 1013: 173-181. The CE-MS technology allows one to determine the presence of some hundreds of polypeptide markers of a sample simultaneously within a short time and in a small volume with high sensitivity. After a sample has been measured, a pattern of the measured polypeptide markers is prepared. This pattern can be compared with reference patterns of sick or healthy subjects. In most cases, it is sufficient to use a limited number of polypeptide markers for the diagnosis of prostate cancer and the differential diagnosis between prostate cancer and BPH. A CE-MS method which includes CE coupled on-line to an ESI-TOF MS device is further preferred.

For CE-MS, the use of volatile solvents is preferred, and it is best to work under essentially salt-free conditions. Examples of suitable solvents include acetonitrile, methanol and the like. The solvents can be diluted with water or admixed with a weak acid (e.g., from 0.1% to 1% formic acid) in order to protonate the analyte, preferably the polypeptides.

By means of capillary electrophoresis, it is possible to separate molecules by their charge and size. Neutral particles will migrate at the speed of the electroosmotic flow upon application of a current, while cations are accelerated towards the cathode, and anions are delayed. The advantage of capillaries in electrophoresis resides in their favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are from 30 to 100 cm. In addition, the capillaries are usually made of plastic-coated silica glass. The capillaries may be both untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the performance or altered meanwhile.

In a preferred method for measuring polypeptide markers, the markers of the sample are separated by means of capillary electrophoresis, then directly ionized and transferred on-line to a mass spectrometer coupled thereto for detection.

In the method according to the disclosure, it is advantageous to use several polypeptide markers for the diagnosis of prostate cancer. In particular, at least three polypeptide markers may be used, for example, markers 1, 2 and 3; 1, 2 and 4; etc.

More preferred is the use of at least 4, 5 or 6 markers.

Even more preferred is the use of at least 11 markers, for example, markers 1 to 11.

Most preferred is the use of all markers listed in Tables 1 or 3.

Several markers may also be used for the differential diagnosis between PCA and BPH. In particular, at least three polypeptide markers can be used, for example, markers 45, 46 and 47; 45, 46 and 48; etc.

More preferred is the use of at least 4, 5 or 6 markers.

Even more preferred is the use of at least 7 markers, for example, markers 1 to 7.

Most preferred is the use of all 27 markers listed in Tables 2 or 4.

In order to determine the probability of the existence of prostate cancer when several markers are used, statistic methods known to the skilled person may be used. For example, the Random Forests method described by Weissinger et al. (Kidney Int., 2004, 65: 2426-2434) may be used by using a computer program such as S-Plus.

### EXAMPLE

### 1. Sample Preparation

For detecting the polypeptide markers for prostate cancer, urine was employed. Urine was withdrawn from healthy donors (control group) as well as from patients suffering from prostate cancer or BPH.

For the subsequent CE-MS measurement, the proteins which are also contained in the urine of patients in a higher concentration, such as albumin and immunoglobulins, had to be separated off by ultrafiltration. Thus, 500 μl of urine was removed and admixed with 2 ml of filtration buffer (4 M urea, 0.1 M NaCl, 0.01% ammonia). This 2.5 ml of sample volume was ultrafiltered (Amicon 30 kDa, Millipore, Bedford, USA). The ultrafiltration was performed at 3000 rpm in a centrifuge until 2 ml of ultrafiltrate was obtained.

The 2 ml of filtrate obtained was then applied to a Pharmacia C-2 column (Pharmacia, Uppsala, Sweden) in order to remove urea, salts and other disturbing components. The bound polypeptides were then eluted from the C-2 column with 50% acetonitrile, 0.5% formic acid in water, and lyophilized. For the CE-MS measurement, the polypeptides were then re-suspended with 20 μl of water (HPLC grade, Merck).

### 2. CE-MS Measurement

The CE-MS measurements were performed with a capillary electrophoresis system from Beckman Coulter (P/ACE MDQ System; Beckman Coulter Inc., Fullerton, USA) and an ESI-TOF mass spectrometer from Bruker (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 30% methanol and 0.5% formic acid in water. For the “sheath flow” on the MS, 30% isopropanol with 0.5% formic acid was used at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With these parameters, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. After the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good signal intensity as possible on the side of the MS, the nebulizer gas was set to the lowest possible value. The voltage applied to the spray needle for generating the electrospray was 3700-4100 V. The remaining settings at the mass spectrometer were optimized for peptide detection according to the manufacturer's protocol. The spectra were recorded over a mass range of m/z 400 to m/z 3000 and accumulated every 3 seconds.

### 3. Standards for the CE Measurement

For checking and calibrating the CE measurement, the following proteins or polypeptides which are characterized by the stated CE migration times under the selected conditions were employed:

| Protein/polypeptide | Migration time |
|---|---|
| Aprotinin (SIGMA, Taufkirchen, DE, Cat. # A1153) | 9.2 min |
| Ribonuclease (SIGMA, Taufkirchen, DE, Cat. # R4875) | 10.9 min |
| Lysozyme (SIGMA, Taufkirchen, DE, Cat. # L7651) | 8.9 min |
| “REV”, Sequence: REVQSKIGYGRQIIS | 15.6 min |
| “ELM”, Sequence: ELMTGELPYSHINNRDQIIFMVGR | 23.4 min |
| “KINCON”, Sequence: TGSLPYSHIGSRDQIIFMVGR | 20.0 min |
| “GIVLY” Sequence: GIVLYELMTGELPYSHIN | 36.8 min |

The proteins/polypeptides were employed at a concentration of 10 pmol/μl each in water.
“REV”, “ELM, “KINCON” and “GIVLY” are synthetic peptides.

The molecular masses of the peptides and the m/z ratios of the individual charge states visible in MS are listed in the following Table:

| | H (mono) | | | | | | |
|---|---|---|---|---|---|---|---|
| | 1.0079 | 1.0079 | 1.0079 | 1.0079 | 1.0079 | 1.0079 | 1.0079 |
| | m/z | | | | | | |
| | Aprotinin | Ribonuclease | Lysozyme | REV | KINCON | ELM | GIVLY |
| | Mono | Mono | Mono | Mono | Mono | Mono | Mono |
| | Mass | Mass | Mass | Mass | Mass | Mass | Mass |
| 0 | 6513.09 | 13681.32 | 14303.88 | 1732.96 | 2333.19 | 2832.41 | 2048.03 |
| 1 | 6514.0979 | 13682.328 | 14304.888 | 1733.9679 | 2334.1979 | 2833.4179 | 2049.0379 |
| 2 | 3257.5529 | 6841.6679 | 7152.9479 | 867.4879 | 1167.6029 | 1417.2129 | 1025.0229 |
| 3 | 2172.0379 | 4561.4479 | 4768.9679 | 578.6612 | 778.7379 | 945.1446 | 683.6846 |
| 4 | 1629.2804 | 3421.3379 | 3576.9779 | 434.2479 | 584.3054 | 709.1104 | 513.0154 |
| 5 | 1303.6259 | 2737.2719 | 2861.7839 | 347.5999 | 467.6459 | 567.4899 | 410.6139 |
| 6 | 1086.5229 | 2281.2279 | 2384.9879 | 289.8346 | 389.8729 | 473.0762 | 342.3462 |
| 7 | 931.4494 | 1955.4822 | 2044.4193 | 248.5736 | 334.3208 | 405.6379 | 293.5836 |
| 8 | 815.1442 | 1711.1729 | 1788.9929 | 217.6279 | 292.6567 | 355.0592 | 257.0117 |
| 9 | 724.6846 | 1521.1546 | 1590.3279 | 193.559 | 260.2512 | 315.7201 | 228.5668 |
| 10 | 652.3169 | 1369.1399 | 1431.3959 | 174.3039 | 234.3269 | 284.2489 | 205.8109 |
| 11 | 593.107 | 1244.7643 | 1301.3606 | 158.5497 | 213.1161 | 258.4997 | 187.1924 |
| 12 | 543.7654 | 1141.1179 | 1192.9979 | 145.4212 | 195.4404 | 237.0421 | 171.6771 |
| 13 | 502.0148 | 1053.4171 | 1101.3063 | 134.3125 | 180.4841 | 218.8856 | 158.5486 |

### Example 4

### Performance of the Markers Described in WO 01/25791

WO 01/25791 mentions eleven markers (see claim 2 thereof), and the accuracy of mass determination is stated to be 0.5%. If it is considered that these are only fragments of semenogelin I, the following numbers of possible fragments of semenogelin are obtained:

| | Number of possible fragments | |
|---|---|---|
| Molecular masses of the biomarker | from seminogelin I | |
| polypeptides for prostate carcinoma | Mass deviation | Mass |
| [g/mol] | 0.5% | deviation 0.03% |
| 2776 | 100 | 8 |
| 4423 | 182 | 16 |
| 4480 | 173 | 8 |
| 5753 | 212 | 11 |
| 6098 | 214 | 7 |
| 6270 | 217 | 19 |
| 6998 | 248 | 12 |
| 7843 | 270 | 11 |
| 8030 | 265 | 23 |
| 8240 | 281 | 12 |
| 8714 | 299 | 23 |

Further, WO 01/25791 discloses markers that speak against the existence of a prostate carcinoma. The number of possible fragments is contained in the following Table:

| Molecular masses of the | | |
|---|---|---|
| biomarker polypeptides | Number of possible fragments | |
| for prostate | from seminogelin I | |
| carcinoma [g/mol] | Mass deviation 0.5% | Mass deviation 0.03% |
| 2095 | 83 | 2 |
| 2276 | 84 | 5 |
| 2530 | 99 | 6 |
| 3030 | 106 | 4 |
| 3038 | 116 | 10 |
| 3224 | 127 | 7 |
| 3600 | 139 | 6 |
| 3835 | 146 | 9 |
| 3915 | 150 | 16 |
| 3933 | 153 | 23 |
| 4175 | 161 | 14 |

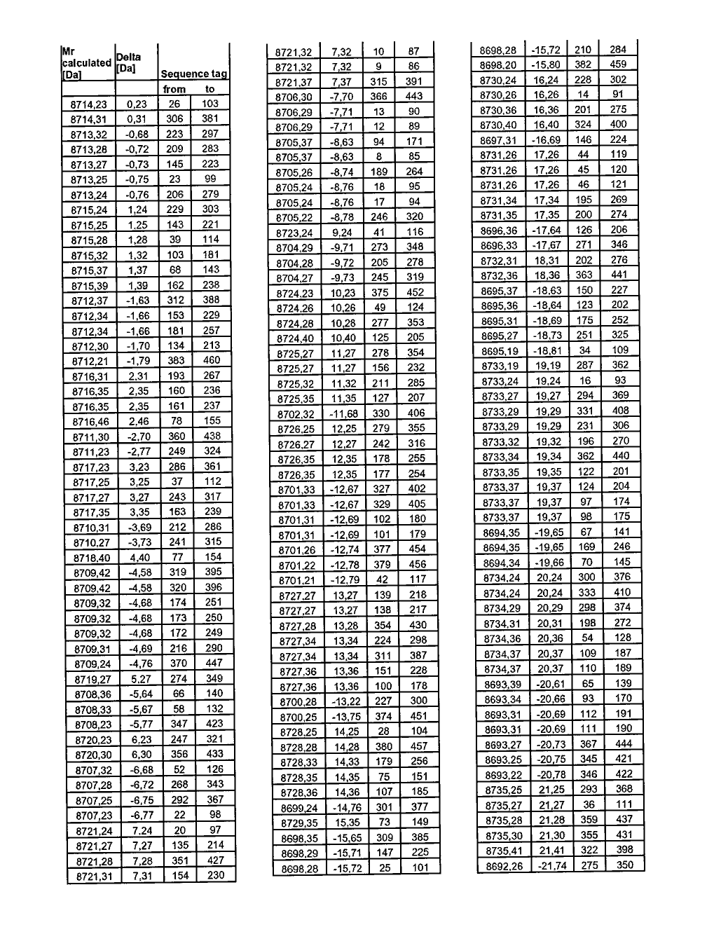

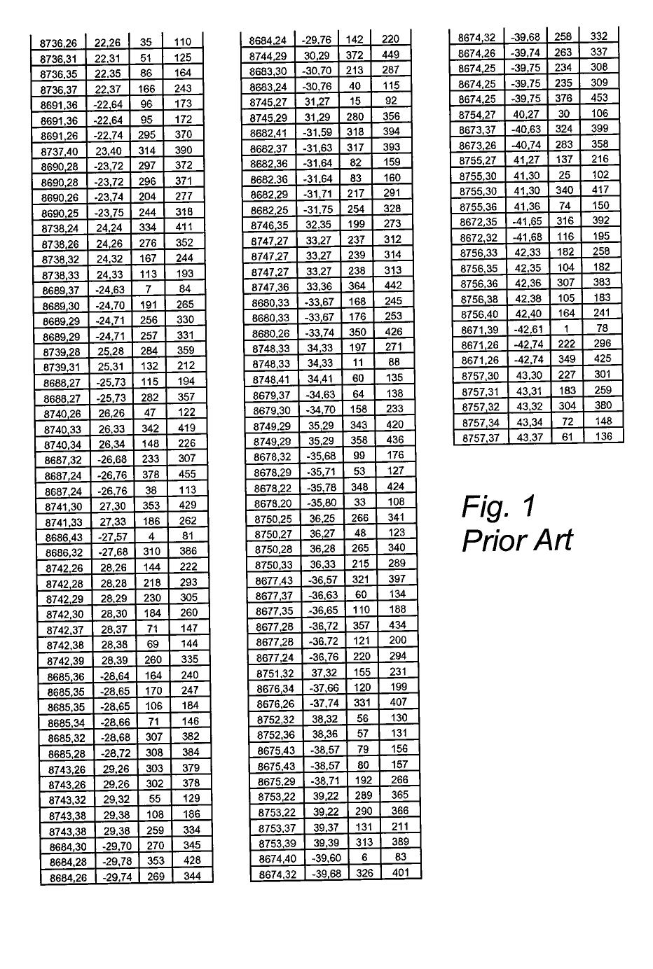

Illustratively for one mass, FIG. 1 shows the large number of sequences that are possible in this weight range.

In the next step, it was tried to analyze the corresponding proteins in urine samples from patients with prostate carcinoma or BPH. Only six markers could be found.

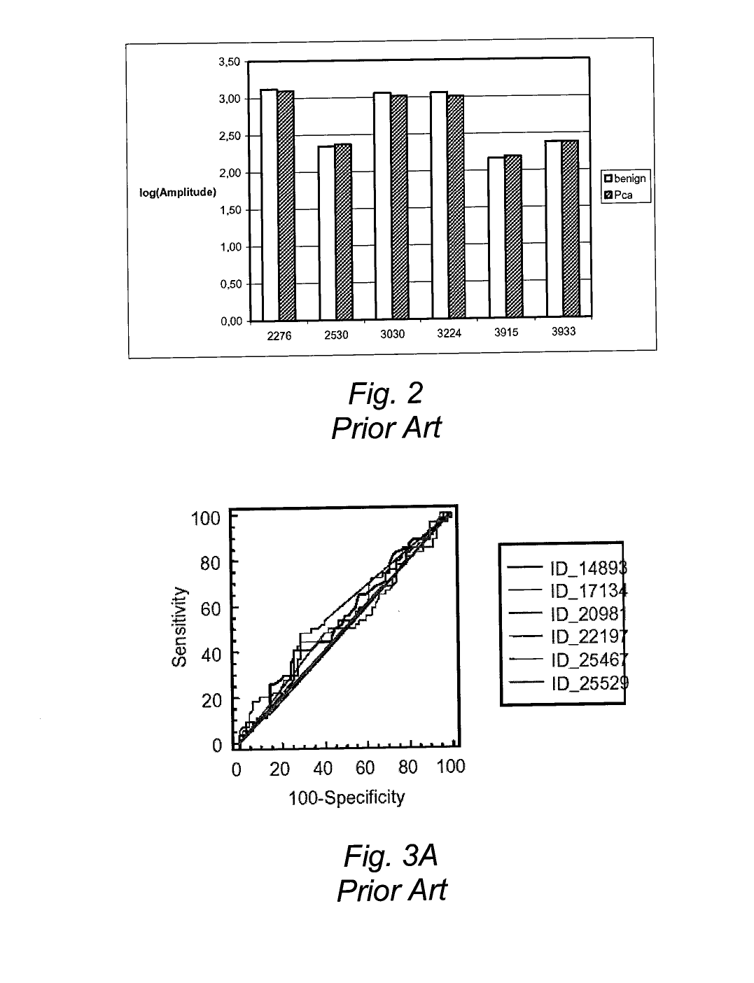

FIG. 2 shows a determination of the amplitudes of the six markers found in this way. It is found that no significant differences occur between prostate carcinoma patients and the control group. Subsequently, the discriminatory value of the biomarkers was examined by means of an ROC (receiver operator characteristic curves) analysis.

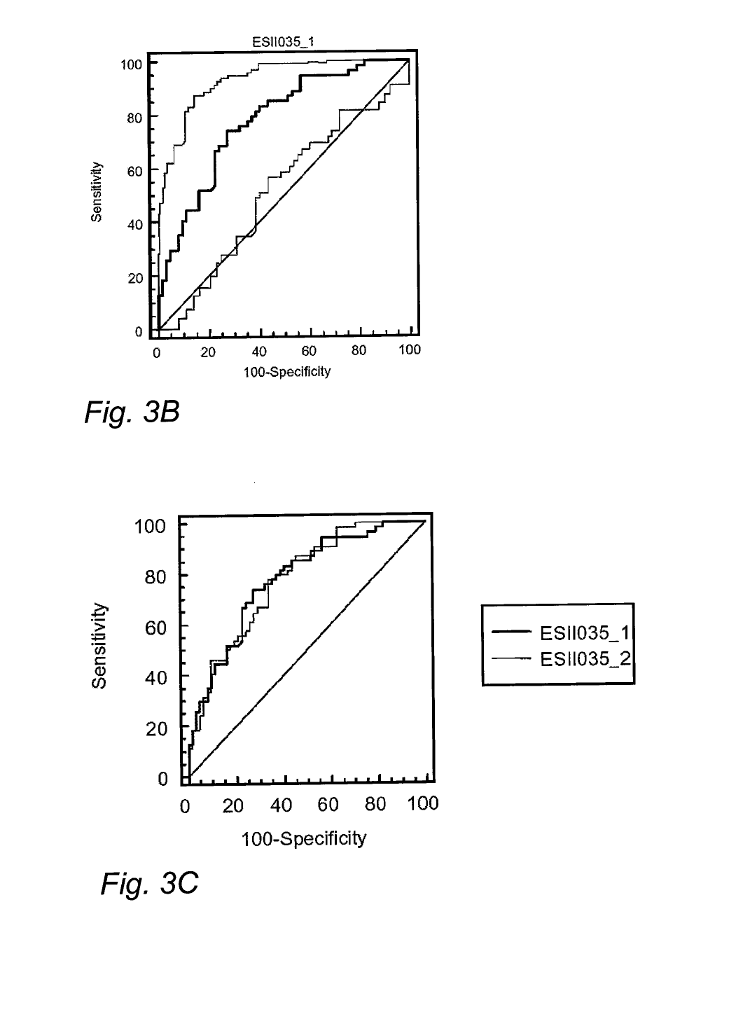

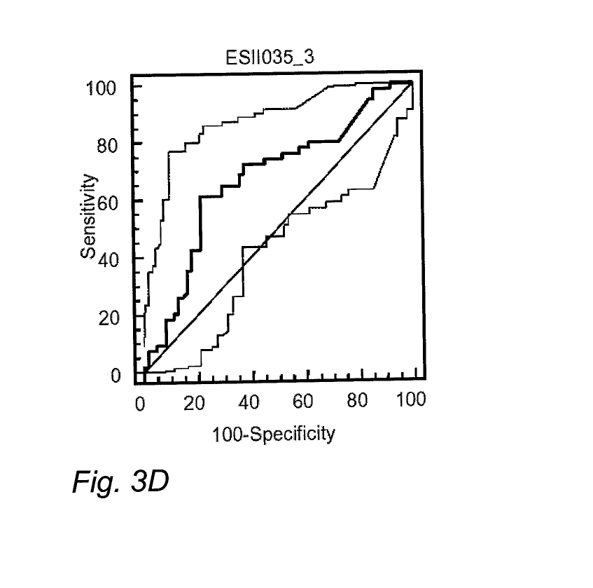

FIG. 3a shows corresponding analyses for six markers that are described in WO 01/25791 and could be found in urine samples.

FIG. 3b shows a corresponding ROC examination of the biomarkers according to the disclosure with Nos. 1 to 78 of the present patent application.

FIG. 3c shows the ROC analysis of a subgroup of the markers according to the disclosure (16 markers). FIG. 3d shows that the significance is clearly higher than that of WO 01/25791 even if only three biomarkers according to the disclosure were used, namely markers 24, 41 and 46.

1. A method for the diagnosis of the probability of the presence of prostate cancer in a subject patient, comprising:
obtaining a urine sample from said subject patient;
purifying said urine sample to remove high concentration proteins;
separating said urine sample into a plurality of polypeptides based on physical characteristics of each of said plurality of polypeptides;
identifying each of said plurality of polypeptides based on said physical characteristics;
comparing said identified plurality of polypeptides to known polypeptide markers obtained from control subjects with prostate cancer and without prostate cancer to obtain a subset of polypeptide markers from said plurality of polypeptides that substantially match the physical characteristics of said known polypeptide markers;
wherein said known polypeptide markers are characterized by physical characteristics comprising the following molecular masses and migration times (CE time):

| | | CE time |
|---|---|---|
| Number | | (min) |
| | Mass (Da) | |
| 1 | 1579.7 | 26.4 |
| 2 | 1991.9 | 17.1 |
| 3 | 1955.9 | 24.8 |
| 4 | 1140.5 | 21.4 |
| 5 | 1677.3 | 7.4 |
| 6 | 10753.1 | 13.6 |
| 7 | 1412.6 | 22.1 |
| 8 | 1636.8 | 27.2 |
| 9 | 1946.9 | 28.4 |
| 10 | 5887.6 | 25.1 |
| 11 | 12407.9 | 22.2 |
| 12 | 1186.6 | 17.4 |
| 13 | 1240.6 | 23.3 |
| 14 | 1326.6 | 24.8 |
| 15 | 3802.1 | 30.1 |
| 16 | 1749.9 | 19.4 |
| 17 | 2216.1 | 31.0 |
| 18 | 1069.5 | 22.7 |
| 19 | 1341.6 | 26.6 |
| 20 | 1353.7 | 21.8 |
| 21 | 1716.8 | 24.6 |
| 22 | 2939.1 | 31.1 |
| 23 | 3002.0 | 19.2 |
| 24 | 1110.4 | 31.5 |
| 25 | 1496.7 | 26.5 |
| 26 | 1825.8 | 28.4 |
| 27 | 1180.5 | 33.9 |
| 28 | 2421.1 | 32.5 |
| 29 | 4345.9 | 30.6 |
| 30 | 2077.1 | 16.9 |
| 31 | 1134.6 | 19.4 |
| 32 | 1444.8 | 13.5 |
| 33 | 1046.5 | 21.9 |
| 34 | 1992.2 | 16.8 |
| 35 | 4069.6 | 21.3 |
| 36 | 1191.5 | 34.5 |
| 37 | 1239.5 | 32.5 |
| 38 | 2191.9 | 17.2 |
| 39 | 1865.8 | 30.0 |
| 40 | 1265.6 | 23.4 |
| 41 | 1936.1 | 10.5 |
| 42 | 911.3 | 31.9 |
| 43 | 1494.7 | 26.4 |
| 44 | 1209.6 | 22.5 |
| 45 | 1193.3 | 34.2 |
| 46 | 1235.4 | 34.3 |
| 47 | 1390.5 | 35.0 |
| 48 | 2292.1 | 23.7 |
| 49 | 2736.3 | 17.1 |
| 50 | 3385.5 | 21.0 |
| 51 | 3945.2 | 21.4 |
| | Masses | |
| | (Da) | |
| 52 | 1636.8 | 27.2 |
| 53 | 1412.6 | 22.1 |
| 54 | 1579.7 | 26.4 |
| 55 | 1390.5 | 35.0 |
| 56 | 1196.6 | 15.8 |
| 57 | 11041.4 | 16.6 |
| 58 | 2971.4 | 17.9 |
| 59 | 3136.6 | 20.0 |
| 60 | 4409.9 | 14.2 |
| 61 | 1494.7 | 26.4 |
| 62 | 1833.9 | 24.2 |
| 63 | 2752.4 | 14.0 |
| 64 | 3515.8 | 15.3 |
| 65 | 1082.5 | 17.7 |
| 66 | 1878.9 | 15.3 |
| 67 | 3593.4 | 14.5 |
| 68 | 1236.7 | 13.3 |
| 69 | 2736.3 | 17.1 |
| 70 | 2973.4 | 20.0 |
| 71 | 1134.6 | 19.4 |
| 72 | 2191.9 | 17.2 |
| 73 | 2205.1 | 25.1 |
| 74 | 3959.7 | 14.0 |
| 75 | 1749.9 | 19.4 |
| 76 | 4069.6 | 21.3 |
| 77 | 2816.3 | 24.7 |
| 78 | 3435.8 | 15.0 |
| | Mass (Da) | |
| 79 | 973.26 | 35.44 |
| 80 | 1128.54 | 25.66 |
| 81 | 1173.58 | 37.47 |
| 82 | 1184.6 | 26.43 |
| 83 | 1290.39 | 30.85 |
| 84 | 1338.66 | 23.96 |
| 85 | 1428.45 | 36.73 |
| 86 | 1450.6 | 37.47 |
| 87 | 1460.71 | 19.83 |
| 88 | 1498.46 | 35.31 |
| 89 | 1526.76 | 23.54 |
| 90 | 1588.77 | 30.24 |
| 91 | 1675.77 | 29.23 |
| 92 | 1703.91 | 33.67 |
| 93 | 1808.87 | 23.72 |
| 94 | 1863.96 | 44.01 |
| 95 | 1867.79 | 33.29 |
| 96 | 1925.9 | 23.2 |
| 97 | 2036.97 | 31.53 |
| 98 | 2114.05 | 31.59 |
| 99 | 2196.11 | 33.16 |
| 100 | 2212.06 | 33.36 |
| 101 | 2257.95 | 36.02 |
| 102 | 2544.06 | 26.14 |
| 103 | 2599.21 | 28.05 |
| 104 | 2761.38 | 21.47 |
| 105 | 3334.17 | 31.01 |
| 106 | 3361.41 | 24.32 |
| 107 | 3426.43 | 27.73 |
| 108 | 3765.51 | 20.18 |
| 109 | 3864.56 | 33.82 |
| 110 | 3870.8 | 33.47 |
| 111 | 4252.03 | 28.77 |
| 112 | 6211.93 | 20.27 |
| 113 | 6650.86 | 25.55 |
| 114 | 6820.37 | 21.03 |
| 115 | 16918.24 | 19.8 |

wherein said CE times are based on capillary electrophoresis using a glass capillary with of an inner diameter of 50 μm and a length of 90 cm with a mobile phase consisting of 30% methanol and 0.5% formic acid in water at a separation voltage of 30 kV, and
wherein said CE times are calibrated relative to the following values:

| Protein/polypeptide | Migration time |
|---|---|
| Ribonuclease | 10.9 min |
| Lysozyme | 8.9 min |
| “REV” | 15.6 min |
| “ELM” | 23.4 min |
| “KINCON” | 20.0 min |
| “GIVLY” | 36.8 min; |

determining if said subset of polypeptide markers comprises at least:
a first biomarker selected from the group consisting of: (1) markers 1 to 44 and 52 to 78 (frequency markers) having a frequency in patients with prostate cancer of from about 0.75 to about 1.0; (2) markers 1 to 44 and 52 to 78 (frequency markers) having a frequency in patients with prostate cancer of from about 0.0 to about 0.25; (3) markers 45 to 51 and 79 to 115 (amplitude markers) having a mean amplitude in patients with prostate cancer of from about 1.5 to about 2.2 times the mean amplitude in patients without prostate cancer; and (4) markers 45 to 51 and 79 to 115 (amplitude markers) having a mean amplitude in patients without prostate cancer of from about 2.3 to about 2.9 times the mean amplitude in patients with prostate cancer,
a second biomarker which is different than said at least first biomarker and is selected from the group consisting of: (1) markers 1 to 44 and 52 to 78 (frequency markers) having a frequency in patients with prostate cancer of from about 0.75 to about 1.0; (2) markers 1 to 44 and 52 to 78 (frequency markers) having a frequency in patients with prostate cancer of from about 0.0 to about 0.25; (3) markers 45 to 51 and 79 to 115 (amplitude markers) having a mean amplitude in patients with prostate cancer of from about 1.5 to about 2.2 times the mean amplitude in patients without prostate cancer; and (4) markers 45 to 51 and 79 to 115 (amplitude markers) having a mean amplitude in patients without prostate cancer of from about 2.3 to about 2.9 times the mean amplitude in patients with prostate cancer, and
a third biomarker is different than said first and second biomarkers and is at least one selected from the group consisting of markers 1-115;
comparing the frequency of presence or amplitude of said at least said first, second and third biomarkers in said urine sample of said subject patient to the frequency of presence or amplitude of the same first, second and third biomarkers from said known polypeptide markers taken from said control subjects;
ranking said subject patient between said control subjects with prostate cancer and without prostate cancer based on the second comparing step; and
diagnosing the probability of the presence of prostate cancer in a subject patient based on said ranking.

2. The method according to claim 1, wherein the frequency values for frequency makers 1-44 and 52 to 78 is based upon the following reference frequency values:

| | Frequency | |
|---|---|---|
| | in Prostate | |
| | Cancer | Frequency |
| | (PCA) | in control |
| Number | group | group |
| 1 | 1 | 0.25 |
| 2 | 0.88 | 0.22 |
| 3 | 1 | 0.35 |
| 4 | 1 | 0.43 |
| 5 | 0.63 | 0.06 |
| 6 | 0.88 | 0.33 |
| 7 | 0.75 | 0.22 |
| 8 | 0.63 | 0.1 |
| 9 | 0.75 | 0.22 |
| 10 | 1 | 0.49 |
| 11 | 0.75 | 0.24 |
| 12 | 0.38 | 0.88 |
| 13 | 0.25 | 0.76 |
| 14 | 0.38 | 0.88 |
| 15 | 0.38 | 0.88 |
| 16 | 0.13 | 0.65 |
| 17 | 0.13 | 0.65 |
| 18 | 0 | 0.53 |
| 19 | 0 | 0.53 |
| 20 | 0.25 | 0.78 |
| 21 | 0.25 | 0.78 |
| 22 | 0.38 | 0.9 |
| 23 | 0.25 | 0.78 |
| 24 | 0.13 | 0.67 |
| 25 | 0.13 | 0.67 |
| 26 | 0.13 | 0.67 |
| 27 | 0 | 0.55 |
| 28 | 0 | 0.55 |
| 29 | 0 | 0.55 |
| 30 | 0.13 | 0.69 |
| 31 | 0.25 | 0.82 |
| 32 | 0.13 | 0.71 |
| 33 | 0 | 0.59 |
| 34 | 0 | 0.59 |
| 35 | 0 | 0.59 |
| 36 | 0 | 0.61 |
| 37 | 0 | 0.61 |
| 38 | 0.25 | 0.86 |
| 39 | 0.13 | 0.75 |
| 40 | 0.25 | 0.9 |
| 41 | 0 | 0.65 |
| 42 | 0 | 0.69 |
| 43 | 0.13 | 0.82 |
| 44 | 0 | 0.71 |
| | | | | | Frequency |
|---|---|---|---|---|---|
| | | | | | for benign |
| | | | | | prostate |
| | | | | Frequency | hyperplasia |
| | Masses | CE time | | for PCA | (BPH) |
| Number | (Da) | (min) | Difference | group | group |
| 52 | 1636.8 | 27.2 | D: 0.63 | 0.63 | 0 |
| 53 | 1412.6 | 22.1 | D: 0.57 | 0.75 | 0.18 |
| 54 | 1579.7 | 26.4 | D: 0.55 | 1 | 0.45 |
| 55 | 1390.5 | 35.0 | D: 0.53 | 0.63 | 0.09 |
| 56 | 1196.6 | 15.8 | D: 0.51 | 0.88 | 0.36 |
| 57 | 11041.4 | 16.6 | D: 0.51 | 0.88 | 0.36 |
| 58 | 2971.4 | 17.9 | D: 0.50 | 0.5 | 0 |
| 59 | 3136.6 | 20.0 | D: −0.50 | 0.5 | 1 |
| 60 | 4409.9 | 14.2 | D: −0.50 | 0.5 | 1 |
| 61 | 1494.7 | 26.4 | D: −0.51 | 0.13 | 0.64 |
| 62 | 1833.9 | 24.2 | D: −0.51 | 0.13 | 0.64 |
| 63 | 2752.4 | 14.0 | D: −0.51 | 0.13 | 0.64 |
| 64 | 3515.8 | 15.3 | D: −0.51 | 0.13 | 0.64 |
| 65 | 1082.5 | 17.7 | D: −0.53 | 0.38 | 0.91 |
| 66 | 1878.9 | 15.3 | D: −0.53 | 0.38 | 0.91 |
| 67 | 3593.4 | 14.5 | D: −0.53 | 0.38 | 0.91 |
| 68 | 1236.7 | 13.3 | D: −0.55 | 0 | 0.55 |
| 69 | 2736.3 | 17.1 | D: −0.55 | 0 | 0.55 |
| 70 | 2973.4 | 20.0 | D: −0.55 | 0 | 0.55 |
| 71 | 1134.6 | 19.4 | D: −0.57 | 0.25 | 0.82 |
| 72 | 2191.9 | 17.2 | D: −0.57 | 0.25 | 0.82 |
| 73 | 2205.1 | 25.1 | D: −0.57 | 0.25 | 0.82 |
| 74 | 3959.7 | 14.0 | D: −0.57 | 0.25 | 0.82 |
| 75 | 1749.9 | 19.4 | D: −0.60 | 0.13 | 0.73 |
| 76 | 4069.6 | 21.3 | D: −0.64 | 0 | 0.64 |
| 77 | 2816.3 | 24.7 | D: −0.73 | 0 | 0.73 |
| 78 | 3435.8 | 15.0 | D: −0.82 | 0 | 0.82 |

3. The method according to claim 1, wherein the amplitude values for amplitude makers 45 to 51 and 79-115 is based upon the following reference amplitude values:

| | | | Mean amplitude in | |
|---|---|---|---|---|
| | | CE | Prostate Cancer | Mean amplitude |
| | Masses | time | (PCA) samples | in control samples |
| Number | (Da) | (min) | (ppm) | (min) |
| 45 | 1193.3 | 34.2 | 24000 | 11000 |
| 46 | 1235.4 | 34.3 | 160 | 370 |
| 47 | 1390.5 | 35.0 | 22000 | 12000 |
| 48 | 2292.1 | 23.7 | 2600 | 1600 |
| 49 | 2736.3 | 17.1 | 120 | 350 |
| 50 | 3385.5 | 21.0 | 2600 | 1700 |
| 51 | 3945.2 | 21.4 | 260 | 120 |
| | | | Mean amplitude | Mean amplitude in |
|---|---|---|---|---|
| | Masses | CE time | in PCA samples | control samples |
| Number | (Da) | (min) | (ppm) | (ppm) |
| 79 | 973.26 | 35.44 | 112.27 | 94.56 |
| 80 | 1128.54 | 25.66 | 104.1 | 135.92 |
| 81 | 1173.58 | 37.47 | 163.08 | 174.37 |
| 82 | 1184.6 | 26.43 | 61.33 | 53.82 |
| 83 | 1290.39 | 30.85 | 425.14 | 360.22 |
| 84 | 1338.66 | 23.96 | 116.85 | 223.77 |
| 85 | 1428.45 | 36.73 | 2325.09 | 1972.91 |
| 86 | 1450.6 | 37.47 | 348.86 | 350.46 |
| 87 | 1460.71 | 19.83 | 204.2 | 216.45 |
| 88 | 1498.46 | 35.31 | 115.26 | 94.66 |
| 89 | 1526.76 | 23.54 | 94.2 | 155.52 |
| 90 | 1588.77 | 30.24 | 504.83 | 565.2 |
| 91 | 1675.77 | 29.23 | 104.37 | 83.95 |
| 92 | 1703.91 | 33.67 | 326.26 | 495.27 |
| 93 | 1808.87 | 23.72 | 189.86 | 198.11 |
| 94 | 1863.96 | 44.01 | 989.35 | 1411.72 |
| 95 | 1867.79 | 33.29 | 117.97 | 108.75 |
| 96 | 1925.9 | 23.2 | 148.34 | 145.13 |
| 97 | 2036.97 | 31.53 | 74.45 | 77.29 |
| 98 | 2114.05 | 31.59 | 135.48 | 121.91 |
| 99 | 2196.11 | 33.16 | 479.36 | 390.89 |
| 100 | 2212.06 | 33.36 | 336.96 | 397.62 |
| 101 | 2257.95 | 36.02 | 365.38 | 567.62 |
| 102 | 2544.06 | 26.14 | 77.74 | 121.23 |
| 103 | 2599.21 | 28.05 | 797.34 | 915.08 |
| 104 | 2761.38 | 21.47 | 744.83 | 616.6 |
| 105 | 3334.17 | 31.01 | 105.87 | 127.64 |
| 106 | 3361.41 | 24.32 | 143.28 | 108.31 |
| 107 | 3426.43 | 27.73 | 149.46 | 116.27 |
| 108 | 3765.51 | 20.18 | 375.75 | 309.53 |
| 109 | 3864.56 | 33.82 | 271.44 | 162.02 |
| 110 | 3870.8 | 33.47 | 71.63 | 78.31 |
| 111 | 4252.03 | 28.77 | 388.07 | 447.93 |
| 112 | 6211.93 | 20.27 | 492.21 | 400.75 |
| 113 | 6650.86 | 25.55 | 233.38 | 232.51 |
| 114 | 6820.37 | 21.03 | 258.88 | 227.62 |
| 115 | 16918.24 | 19.8 | 605.64 | 324.52 |

4. The method according to claim 1, wherein at least four polypeptide markers are used.

5. The method according to claim 1, wherein at least ten polypeptide markers are used.

6. A method for performing a differential diagnosis between prostate cancer (PCA) and benign prostate hyperplasia (BPH) in a subject patient, comprising:
obtaining a urine sample from said subject patient;
purifying said urine sample to remove high concentration proteins;
separating said urine sample into a plurality of polypeptides based on physical characteristics of each of said plurality of polypeptides;
identifying each of said plurality of polypeptides based on said physical characteristics;
comparing said plurality of polypeptides to known polypeptide markers obtained from control subjects with prostate cancer and with benign prostate hyperplasia to obtain a subset of polypeptide markers from said plurality of polypeptides that substantially match the physical characteristics of said known polypeptide markers;
wherein said known polypeptide markers are characterized by physical characteristics comprising the following molecular masses and migration times (CE time):

| | Masses | CE time |
|---|---|---|
| Number | (Da) | (min) |
| 52 | 1636.8 | 27.2 |
| 53 | 1412.6 | 22.1 |
| 54 | 1579.7 | 26.4 |
| 55 | 1390.5 | 35.0 |
| 56 | 1196.6 | 15.8 |
| 57 | 11041.4 | 16.6 |
| 58 | 2971.4 | 17.9 |
| 59 | 3136.6 | 20.0 |
| 60 | 4409.9 | 14.2 |
| 61 | 1494.7 | 26.4 |
| 62 | 1833.9 | 24.2 |
| 63 | 2752.4 | 14.0 |
| 64 | 3515.8 | 15.3 |
| 65 | 1082.5 | 17.7 |
| 66 | 1878.9 | 15.3 |
| 67 | 3593.4 | 14.5 |
| 68 | 1236.7 | 13.3 |
| 69 | 2736.3 | 17.1 |
| 70 | 2973.4 | 20.0 |
| 71 | 1134.6 | 19.4 |
| 72 | 2191.9 | 17.2 |
| 73 | 2205.1 | 25.1 |
| 74 | 3959.7 | 14.0 |
| 75 | 1749.9 | 19.4 |
| 76 | 4069.6 | 21.3 |
| 77 | 2816.3 | 24.7 |
| 78 | 3435.8 | 15.0, |

wherein said CE times are based on capillary electrophoresis using a glass capillary with an inner diameter of 50 and a length of 90 cm with a mobile phase consisting of 30% methanol and 0.5% formic acid in water at a separation voltage of 30 kV, and
wherein said CE times are calibrated relative to the following values:

| Protein/polypeptide | Migration time |
|---|---|
| Aprotinin | 9.2 min |
| Ribonuclease | 10.9 min |
| Lysozyme | 8.9 min |
| “REV” | 15.6 min |
| “ELM” | 23.4 min |
| “KINCON” | 20.0 min |
| “GIVLY” | 36.8 min; |

determining if said subset of polypeptide markers comprises at least:
a first biomarker selected from the group consisting of: (1) markers 52 to 78 (frequency markers) having a frequency in patients with benign prostate hyperplasia of from about 0.55 to about 1.0 and a frequency in patients with prostate cancer of less than about 0.55; and (2) markers 52-78 (frequency markers) having a frequency in patients with benign prostate hyperplasia from about 0.0 to about 0.45;
a second biomarker which is different than said at least first biomarker and is selected from the group consisting of: (1) markers 52 to 78 (frequency markers) having a frequency in patients with benign prostate hyperplasia of from about 0.55 to about 1.0 and a frequency in patients with prostate cancer of less than about 0.55; and (2) markers 52-78 (frequency markers) having a frequency in patients with benign prostate hyperplasia from about 0.0 to about 0.45; and
a third biomarker is different than said first and second biomarkers and is at least one selected from the group consisting of markers 52 to 78;
comparing the frequency of presence of said at least said first, second and third biomarkers in said urine sample of said subject patient to the frequency of presence of the same first, second and third biomarkers from said known polypeptide markers taken from said control subjects,
ranking said subject patient between said control subjects with benign prostate hyperplasia and without benign prostate hyperplasia based on the second comparing step; and
performing said differential diagnosis based on said ranking.

7. The method according to claim 6, wherein the frequency values for frequency makers 52 to 78 is based upon the following reference values:

| | | Frequency |
|---|---|---|
| | | in benign |
| | | prostate |
| | Frequency | hyperplasia |
| | in PCA | (BPH) |
| Number | group | group |
| 52 | 0.63 | 0 |
| 53 | 0.75 | 0.18 |
| 54 | 1 | 0.45 |
| 55 | 0.63 | 0.09 |
| 56 | 0.88 | 0.36 |
| 57 | 0.88 | 0.36 |
| 58 | 0.5 | 0 |
| 59 | 0.5 | 1 |
| 60 | 0.5 | 1 |
| 61 | 0.13 | 0.64 |
| 62 | 0.13 | 0.64 |
| 63 | 0.13 | 0.64 |
| 64 | 0.13 | 0.64 |
| 65 | 0.38 | 0.91 |
| 66 | 0.38 | 0.91 |
| 67 | 0.38 | 0.91 |
| 68 | 0 | 0.55 |
| 69 | 0 | 0.55 |
| 70 | 0 | 0.55 |
| 71 | 0.25 | 0.82 |
| 72 | 0.25 | 0.82 |
| 73 | 0.25 | 0.82 |
| 74 | 0.25 | 0.82 |
| 75 | 0.13 | 0.73 |
| 76 | 0 | 0.64 |
| 77 | 0 | 0.73 |
| 78 | 0 | 0.82 |

8. The method according to claim 6, wherein at least four polypeptide markers are used.

9. The method according to claim 6, wherein at least ten polypeptide markers are used.

10. A method for the diagnosis of the probability of the presence of prostate cancer in a subject patient, comprising:
obtaining a urine sample from said subject patient;
purifying said urine sample to remove high concentration proteins;
separating said urine sample by CE/MS into a plurality of polypeptides based on physical characteristics comprised of charge and size of each of said plurality of polypeptides;
identifying each of said plurality of polypeptides based on said physical characteristics;
comparing said identified plurality of polypeptides to known polypeptide markers obtained from control subjects with prostate cancer and without prostate cancer to obtain a subset of polypeptide markers from said plurality of polypeptides that substantially match the physical characteristics of said known polypeptide markers;
wherein said known polypeptide markers are characterized by physical characteristics comprising the following molecular masses and migration times (CE time):

| | | CE |
|---|---|---|
| | | time |
| Number | | (min) |
| | Mass (Da) | |
| 1 | 1579.7 | 26.4 |
| 2 | 1991.9 | 17.1 |
| 3 | 1955.9 | 24.8 |
| 4 | 1140.5 | 21.4 |
| 5 | 1677.3 | 7.4 |
| 6 | 10753.1 | 13.6 |
| 7 | 1412.6 | 22.1 |
| 8 | 1636.8 | 27.2 |
| 9 | 1946.9 | 28.4 |
| 10 | 5887.6 | 25.1 |
| 11 | 12407.9 | 22.2 |
| 12 | 1186.6 | 17.4 |
| 13 | 1240.6 | 23.3 |
| 14 | 1326.6 | 24.8 |
| 15 | 3802.1 | 30.1 |
| 16 | 1749.9 | 19.4 |
| 17 | 2216.1 | 31.0 |
| 18 | 1069.5 | 22.7 |
| 19 | 1341.6 | 26.6 |
| 20 | 1353.7 | 21.8 |
| 21 | 1716.8 | 24.6 |
| 22 | 2939.1 | 31.1 |
| 23 | 3002.0 | 19.2 |
| 24 | 1110.4 | 31.5 |
| 25 | 1496.7 | 26.5 |
| 26 | 1825.8 | 28.4 |
| 27 | 1180.5 | 33.9 |
| 28 | 2421.1 | 32.5 |
| 29 | 4345.9 | 30.6 |
| 30 | 2077.1 | 16.9 |
| 31 | 1134.6 | 19.4 |
| 32 | 1444.8 | 13.5 |
| 33 | 1046.5 | 21.9 |
| 34 | 1992.2 | 16.8 |
| 35 | 4069.6 | 21.3 |
| 36 | 1191.5 | 34.5 |
| 37 | 1239.5 | 32.5 |
| 38 | 2191.9 | 17.2 |
| 39 | 1865.8 | 30.0 |
| 40 | 1265.6 | 23.4 |
| 41 | 1936.1 | 10.5 |
| 42 | 911.3 | 31.9 |
| 43 | 1494.7 | 26.4 |
| 44 | 1209.6 | 22.5 |
| 45 | 1193.3 | 34.2 |
| 46 | 1235.4 | 34.3 |
| 47 | 1390.5 | 35.0 |
| 48 | 2292.1 | 23.7 |
| 49 | 2736.3 | 17.1 |
| 50 | 3385.5 | 21.0 |
| 51 | 3945.2 | 21.4 |
| | Masses | |
| | (Da) | |
| 52 | 1636.8 | 27.2 |
| 53 | 1412.6 | 22.1 |
| 54 | 1579.7 | 26.4 |
| 55 | 1390.5 | 35.0 |
| 56 | 1196.6 | 15.8 |
| 57 | 11041.4 | 16.6 |
| 58 | 2971.4 | 17.9 |
| 59 | 3136.6 | 20.0 |
| 60 | 4409.9 | 14.2 |
| 61 | 1494.7 | 26.4 |
| 62 | 1833.9 | 24.2 |
| 63 | 2752.4 | 14.0 |
| 64 | 3515.8 | 15.3 |
| 65 | 1082.5 | 17.7 |
| 66 | 1878.9 | 15.3 |
| 67 | 3593.4 | 14.5 |
| 68 | 1236.7 | 13.3 |
| 69 | 2736.3 | 17.1 |
| 70 | 2973.4 | 20.0 |
| 71 | 1134.6 | 19.4 |
| 72 | 2191.9 | 17.2 |
| 73 | 2205.1 | 25.1 |
| 74 | 3959.7 | 14.0 |
| 75 | 1749.9 | 19.4 |
| 76 | 4069.6 | 21.3 |
| 77 | 2816.3 | 24.7 |
| 78 | 3435.8 | 15.0 |
| | Mass (Da) | |
| 79 | 973.26 | 35.44 |
| 80 | 1128.54 | 25.66 |
| 81 | 1173.58 | 37.47 |
| 82 | 1184.6 | 26.43 |
| 83 | 1290.39 | 30.85 |
| 84 | 1338.66 | 23.96 |
| 85 | 1428.45 | 36.73 |
| 86 | 1450.6 | 37.47 |
| 87 | 1460.71 | 19.83 |
| 88 | 1498.46 | 35.31 |
| 89 | 1526.76 | 23.54 |
| 90 | 1588.77 | 30.24 |
| 91 | 1675.77 | 29.23 |
| 92 | 1703.91 | 33.67 |
| 93 | 1808.87 | 23.72 |
| 94 | 1863.96 | 44.01 |
| 95 | 1867.79 | 33.29 |
| 96 | 1925.9 | 23.2 |
| 97 | 2036.97 | 31.53 |
| 98 | 2114.05 | 31.59 |
| 99 | 2196.11 | 33.16 |
| 100 | 2212.06 | 33.36 |
| 101 | 2257.95 | 36.02 |
| 102 | 2544.06 | 26.14 |
| 103 | 2599.21 | 28.05 |
| 104 | 2761.38 | 21.47 |
| 105 | 3334.17 | 31.01 |
| 106 | 3361.41 | 24.32 |
| 107 | 3426.43 | 27.73 |
| 108 | 3765.51 | 20.18 |
| 109 | 3864.56 | 33.82 |
| 110 | 3870.8 | 33.47 |
| 111 | 4252.03 | 28.77 |
| 112 | 6211.93 | 20.27 |
| 113 | 6650.86 | 25.55 |
| 114 | 6820.37 | 21.03 |
| 115 | 16918.24 | 19.8, |

wherein said CE times are based on capillary electrophoresis using a glass capillary with of an inner diameter of 50 μm and a length of 90 cm with a mobile phase consisting of 30% methanol and 0.5% formic acid in water at a separation voltage of 30 kV, and
wherein said CE times are calibrated relative to the following values:

| Protein/polypeptide | Migration time |
|---|---|
| Ribonuclease | 10.9 min |
| Lysozyme | 8.9 min |
| “REV” | 15.6 min |
| “ELM” | 23.4 min |
| “KINCON” | 20.0 min |
| “GIVLY” | 36.8 min; |

determining if said subset of polypeptide markers comprises at least:
a first biomarker selected from the group consisting of: (1) markers 1 to 44 and 52 to 78 (frequency markers) having a frequency in patients with prostate cancer of from about 0.75 to about 1.0; (2) markers 1 to 44 and 52 to 78 (frequency markers) having a frequency in patients with prostate cancer of from about 0.0 to about 0.25; (3) markers 45 to 51 and 79 to 115 (amplitude markers) having a mean amplitude in patients with prostate cancer of from about 1.5 to about 2.2 times the mean amplitude in patients without prostate cancer; and (4) markers 45 to 51 and 79 to 115 (amplitude markers) having a mean amplitude in patients without prostate cancer of from about 2.3 to about 2.9 times the mean amplitude in patients with prostate cancer,
a second biomarker which is different than said at least first biomarker and is selected from the group consisting of: (1) markers 1 to 44 and 52 to 78 (frequency markers) having a frequency in patients with prostate cancer of from about 0.75 to about 1.0; (2) markers 1 to 44 and 52 to 78 (frequency markers) having a frequency in patients with prostate cancer of from about 0.0 to about 0.25; (3) markers 45 to 51 and 79 to 115 (amplitude markers) having a mean amplitude in patients with prostate cancer of from about 1.5 to about 2.2 times the mean amplitude in patients without prostate cancer; and (4) markers 45 to 51 and 79 to 115 (amplitude markers) having a mean amplitude in patients without prostate cancer of from about 2.3 to about 2.9 times the mean amplitude in patients with prostate cancer, and
a third biomarker is different than said first and second biomarkers and is at least one selected from the group consisting of markers 1-115;
comparing the frequency of presence or amplitude of said at least said first, second and third biomarkers in said urine sample of said subject patient to the frequency of presence or amplitude of the same first, second and third biomarkers from said known polypeptide markers taken from said control subjects;
ranking said subject patient between said control subjects with prostate cancer and without prostate cancer based on the second comparing step; and
diagnosing the probability of the presence of prostate cancer in a subject patient based on said ranking.

11. A method for performing a differential diagnosis between prostate cancer (PCA) and benign prostate hyperplasia (BPH) in a subject patient, comprising:
obtaining a urine sample from said subject patient;
purifying said urine sample to remove high concentration proteins;
separating said urine sample by CE/MS into a plurality of polypeptides based on physical characteristics comprised of charge and size of each of said plurality of polypeptides;
identifying each of said plurality of polypeptides based on said physical characteristics;
comparing said plurality of polypeptides to known polypeptide markers obtained from control subjects with prostate cancer and with benign prostate hyperplasia to obtain a subset of polypeptide markers from said plurality of polypeptides that substantially match the physical characteristics of said known polypeptide markers;
wherein said known polypeptide markers are characterized by physical characteristics comprising the following molecular masses and migration times (CE time):

| | Masses | CE time |
|---|---|---|
| Number | (Da) | (min) |
| 52 | 1636.8 | 27.2 |
| 53 | 1412.6 | 22.1 |
| 54 | 1579.7 | 26.4 |
| 55 | 1390.5 | 35.0 |
| 56 | 1196.6 | 15.8 |
| 57 | 11041.4 | 16.6 |
| 58 | 2971.4 | 17.9 |
| 59 | 3136.6 | 20.0 |
| 60 | 4409.9 | 14.2 |
| 61 | 1494.7 | 26.4 |
| 62 | 1833.9 | 24.2 |
| 63 | 2752.4 | 14.0 |
| 64 | 3515.8 | 15.3 |
| 65 | 1082.5 | 17.7 |
| 66 | 1878.9 | 15.3 |
| 67 | 3593.4 | 14.5 |
| 68 | 1236.7 | 13.3 |
| 69 | 2736.3 | 17.1 |
| 70 | 2973.4 | 20.0 |
| 71 | 1134.6 | 19.4 |
| 72 | 2191.9 | 17.2 |
| 73 | 2205.1 | 25.1 |
| 74 | 3959.7 | 14.0 |
| 75 | 1749.9 | 19.4 |
| 76 | 4069.6 | 21.3 |
| 77 | 2816.3 | 24.7 |
| 78 | 3435.8 | 15.0, |

wherein said CE times are based on capillary electrophoresis using a glass capillary with an inner diameter of 50 and a length of 90 cm with a mobile phase consisting of 30% methanol and 0.5% formic acid in water at a separation voltage of 30 kV, and
wherein said CE times are calibrated relative to the following values:

| Protein/polypeptide | Migration time |
|---|---|
| Aprotinin | 9.2 min |
| Ribonuclease | 10.9 min |
| Lysozyme | 8.9 min |
| “REV” | 15.6 min |
| “ELM” | 23.4 min |
| “KINCON” | 20.0 min |
| “GIVLY” | 36.8 min; |

determining if said subset of polypeptide markers comprises at least:
a first biomarker selected from the group consisting of: (1) markers 52 to 78 (frequency markers) having a frequency in patients with benign prostate hyperplasia of from about 0.55 to about 1.0 and a frequency in patients with prostate cancer of less than about 0.55; and (2) markers 52-78 (frequency markers) having a frequency in patients with benign prostate hyperplasia from about 0.0 to about 0.45;
a second biomarker which is different than said at least first biomarker and is selected from the group consisting of: (1) markers 52 to 78 (frequency markers) having a frequency in patients with benign prostate hyperplasia of from about 0.55 to about 1.0 and a frequency in patients with prostate cancer of less than about 0.55; and (2) markers 52-78 (frequency markers) having a frequency in patients with benign prostate hyperplasia from about 0.0 to about 0.45; and
a third biomarker is different than said first and second biomarkers and is at least one selected from the group consisting of markers 52 to 78;
comparing the frequency of presence of said at least said first, second and third biomarkers in said urine sample of said subject patient to the frequency of presence of the same first, second and third biomarkers from said known polypeptide markers taken from said control subjects,
ranking said subject patient between said control subjects with benign prostate hyperplasia and without benign prostate hyperplasia based on the second comparing step; and
performing said differential diagnosis based on said ranking.


SEQUENCE LISTING (4 sequences)

SEQ ID NO: 1 - protein, 15 aa, artificial sequence
  description of artificial sequence synthetic peptide
REVQSKIGYGRQIIS

SEQ ID NO: 2 - protein, 24 aa, artificial sequence
  description of artificial sequence synthetic peptide
ELMTGELPYSHINNRDQIIFMVGR

SEQ ID NO: 3 - protein, 21 aa, artificial sequence
  description of artificial sequence synthetic peptide
TGSLPYSHIGSRDQIIFMVGR

SEQ ID NO: 4 - protein, 18 aa, artificial sequence
  description of artificial sequence synthetic peptide
GIVLYELMTGELPYSHIN